(19) United States
(12) Patent Application Publication
(10) Pub. No.: US 2007/0010005 A1
(43) Pub. Date: Jan. 11, 2007

(54) Neonatal support system and related devices and methods of use

(76) Inventor: James Sitzmann, Zionsville, IN (US)

Correspondence Address: NAMAN, HOWELL, SMITH & LEE, P.O. BOX 1470, WACO, TX, 76703, US

(21) Appl. No.: 11/178,248

(22) Filed: Jul. 8, 2005

(51) Int. Cl. C12M 1/00; A01N 1/02

(52) U.S. Cl. 435/283.1; 435/1.1; 435/289.1

(57) ABSTRACT

In certain embodiments, the present invention relates to a life support system and related devices and systems and methods of use for such devices and systems. In some of these embodiments, the invention relates to a support system for a neonate or fetus and related methods of use of such a system.

### FIELD OF THE INVENTION

In certain embodiments, the present invention relates to a life support system and related devices and systems and methods of use for such devices and systems. More particularly, the invention relates to a support system for a neonate or fetus and related methods of use of such a system.

### BACKGROUND OF THE INVENTION

The presently available systems for neonatal support all have certain inherent disadvantages, including, without limitation, their being limited for use in viable neonates. Generally, the presently available systems for neonatal support provide oxygen exchange through normal or assisted respiration of the neonate. This requires that the lungs of the neonate be sufficiently developed to enable oxygen exchange.

Newborn children may be delivered prematurely for a variety of reasons. Such reasons may include having twins or medical conditions such as trauma or infection of the mother or developing fetus, physiological abnormalities, preeclampsia, or hydramniosis, to name a few. When children are born prematurely, they often have special needs in order to sustain life and promote proper development.

Viability of the prematurely born child often depends on how well-developed the child is at the time of birth. Survival rates generally increase with increased development. Allen et al. previously reported that between 23 and 25 weeks following conception, viability rose from 15 to 79 percent (according to records from a limited group of births). See M. Allen et. al., The Limits of Viability. New England Journal of Medicine. Nov. 25, 1993: Vol. 329, No. 22. For those children born before or even during this window of viability, a need exists for a support system capable of consistently sustaining life and development.

### SUMMARY OF THE INVENTION

One object of the invention is to provide a life support system or apparatus that is capable of sustaining a fetus or prematurely-born neonate outside of a womb.

A further object of the invention is to provide a life support system that is capable of sustaining an aborted fetus, especially where that fetus has an intact cranium.

A further object of the invention is provide a method for sustaining the life of a neonate or fetus outside of a womb.

A further object of the invention is to provide a connection apparatus for connecting to one or both of an umbilical vein and an umbilical artery.

A further object of the invention is to provide a fluid handling system for providing one or both of oxygen and nutrients to the blood of a fetus or neonate.

In one embodiment of the invention, an apparatus for supporting a neonate or a fetus is provided that includes a fluid tank. The fluid tank is at least partially filled with a simulated amniotic fluid. The simulated amniotic fluid is one that mimics the action of amniotic fluid in a womb. The apparatus also includes a dialysis system and an oxygenator. The dialysis system is configured to remove waste from the simulated amniotic fluid. The oxygenator is configured to oxygenate blood circulating through the neonate or fetus. The apparatus may also include a nutritional feeder. The nutritional feeder is configured to deliver at least one nutrient to the neonate or fetus.

According another embodiment of the invention, a method for sustaining the life of a neonate or fetus outside of a mother's womb is provided. The method includes placing the neonate or fetus in a life support system, where the life support system includes a tank at least partially filled with a simulated amniotic fluid. The method also includes a step of providing the fetus or neonate with life support sufficient to sustain the life of the neonate or fetus. The step of providing the fetus or neonate with life support may include one or all of the following: removing waste from the simulated amniotic fluid, oxygenating the blood of the neonate or fetus, and supplementing the blood of the neonate or fetus with nutritionally valuable materials

In accordance with another embodiment of the invention, a connection apparatus for providing a connection to one or both of an umbilical artery and an umbilical vein is provided. The connection apparatus includes an arterial intake element that is configured to be connected to an umbilical artery. The connection apparatus also includes a venous return element, that is configured to be connected to a venous artery. The connection apparatus may also include a circulatory pump configured to cause fluid to flow through one or both of the arterial intake element and the venous return element.

In yet another embodiment of the invention, a fluid handling system is provided. The fluid handling system preferably includes a nutritional feeder, that is configured to deliver at least one nutrient to said fluid. The fluid handling system may also include an oxygenator configured to oxygenate a fluid.

Other objects, advantages and novel features of the present invention will become apparent from the following detailed description of the invention when considered in conjunction with the accompanying drawings.

### BRIEF DESCRIPTION OF THE DRAWINGS

FIG. 1 depicts a life support system in accordance with one embodiment of the invention.

FIG. 2 shows a perspective view of a connection device in accordance with an embodiment of the invention.

FIG. 3 depicts a fluid handling apparatus for use in accordance with an embodiment of the invention.

### DETAILED DESCRIPTION OF PREFERRED EMBODIMENTS

The present invention may be understood by reference to the following detailed description of particular embodiments of the invention and of the specific examples. The terminology used herein is for the purpose of describing particular embodiments only and is not intended to be limiting.

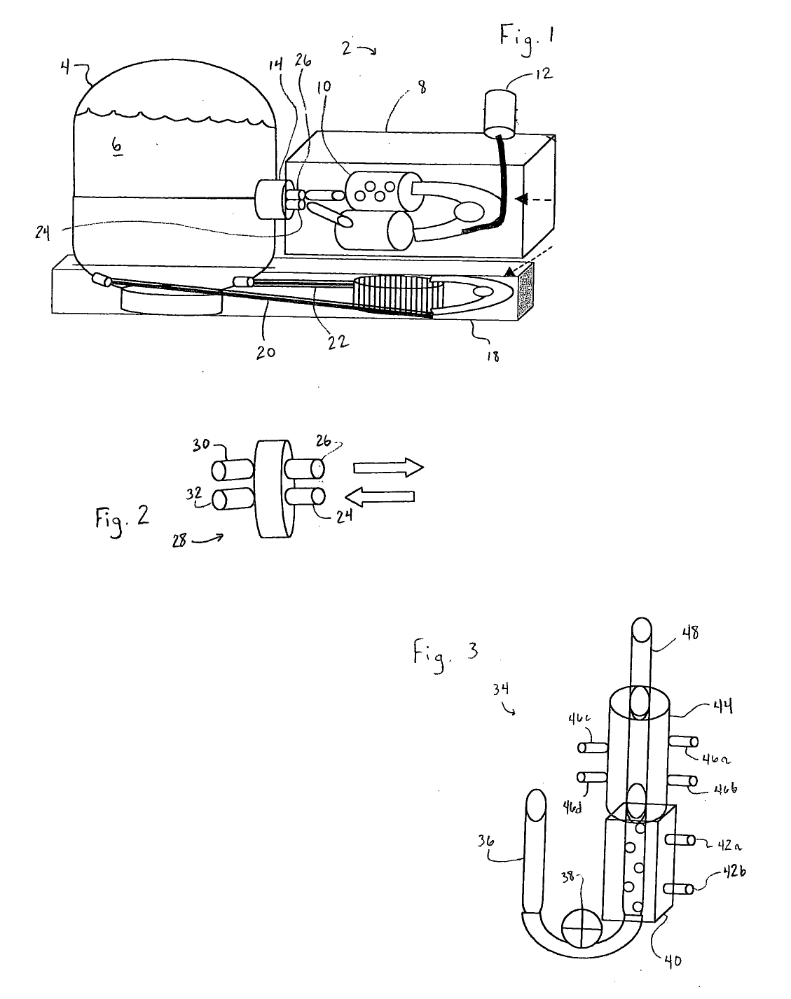

FIG. 1 depicts a system 2 and its component which interact with and provide all elements of essential life support for a neonate or fetus. In a preferred embodiment, the life support system 2 for a neonate or fetus will even support aborted fetuses with intact craniums. The system includes a fluid tank 4 that is at least partially filled with a simulated amniotic fluid 6 that mimics the action of amniotic fluid in the womb. Thus, the simulated amniotic fluid 6 has the same electrolyte, protein content, osmolality, and temperature of amniotic fluid. An oxygenator 8 is provided to continuously replenish the oxygen supply of blood being supplied to the fetus or neonate. The oxygenator 8 includes an oxygen transport apparatus 10 to deliver oxygen to the blood circulating through the oxygenator 8. Nutrition may be provided to the fetus or neonate through a parenteral nutrition feeder 12 which may be associated with the oxygenator 8. The umbilical artery and umbilical vein of the fetus or neonate may be attached by microscopic surgery to the circulatory pump 14, either directly or indirectly, so that the circulatory pump 14 simulates the venous and arterial blood flow the fetus or neonate would normally experience within the womb. In this way, the blood may be continuously pumped through the oxygenator 8.

The simulated amniotic fluid 6 passes out of the fluid tank 4 into a dialysis system 18. The dialysis system 18 reproduces the function of material kidneys, removing one or more waste materials from the simulated amniotic fluid 6. An intake element 20 and exhaust element 22 are used to transport the simulated amniotic fluid 6 into and out of the amniotic dialysis system 18. The dialysis system 18 is configured to drive the flow of the simulated amniotic fluid 6 through the functional portions of the dialysis system 18. Further, the dialysis system 18 may include a heater and a thermostat (not shown) which operate to maintain a constant temperature of the simulated amniotic fluid 6.

The fluid tank 4 may be constructed from a variety of suitable materials, including [what materials might the fluid tank be constructed from]. Further, the fluid tank may be insulated. The fluid tank is of sufficient size to hold a fetus or neonate, and allows for the fetus or neonate to grow into a fully viable baby. The fluid 6 provided in the fluid tank 4 is selected to simulate the action and functionality of amniotic fluid in a mother's womb. Thus, the fluid 6 mimics the action of amniotic fluid in a womb.

[Does a Sterilizer Need to be Provided for the Simulated Amniotic Fluid?]

The nutritional feeder 12 is configured to provide the neonate or fetus with adequate protein, glucose, lipids, elements and vitamins which are essential for and which promote proper survival and growth. In one embodiment, the nutritional feeder 12 is a bag or other fluid container provided with a supply line which supplies nutritional fluid from the nutritional feeder 12 into the blood flowing through the oxygenator 8. Alternatively, the nutritional feeder 12 may be incorporated as part of the oxygenator 8 such that blood flowing through the oxygenator 8 also flows through the nutritional feeder 12.

The oxygenator 8 is preferably configured so that blood flowing through the fetus or neonate flows through the oxygenator 8 similar to the manner in which blood would flow from a fetus to the placenta as the fetus develops during a typical pregnancy. The oxygen transport apparatus 10 of the oxygenator 8 may also be configured to transfer wastes from the blood of the developing fetus or neonate. In yet another embodiment, the oxygen transport apparatus 10 may be configured to deliver nutrients to the fetus or neonate.

FIG. 2 depicts connection apparatus 28 that may be used and associated with the life support system 2 of FIG. 1. The connection apparatus 28 may be used in place of or may be configured as part of circulatory pump 14 of FIG. 1. A venous element 30 extends from the connection apparatus 28 into the fluid tank 4 of FIG. 1. Venous piping 26 is a part of the venous component of the connection apparatus 28 and supplies blood to the oxygenator 8 and nutrition feeder 12 shown in FIG. 1. Blood returns to the fetus or neonate through the arterial component of the connection apparatus 28. The arterial component includes arterial piping 24 that extends toward and may be a part of the oxygenator 8 of FIG. 1. The arterial component also includes an arterial element 32 that extends into the fluid tank 4 of FIG. 1. The arterial element 32 and venous element 30 which extend into the fluid tank 4 of FIG. 1 are preferably configured so that they may be attached to the respective umbilical artery and umbilical vein of the fetus or neonate through microscopic surgery. In this manner the connection apparatus 28 operates to simulate the blood flow a fetus typically experiences in a womb.

The connection apparatus 28 may also include a circulatory pump which facilitates and drives, at least in part, the flow of blood through the system. Alternatively, the circulatory pump may be provided elsewhere in the system; for instance, to facilitate easy replacement of the circulatory pump, or to more accurately replicate the flow of blood to a fetus as driven by a pregnant woman's heart.

FIG. 3 depicts another configuration 34 for the blood oxygenator 40 and nutrition feeder 44 arrangement. In the configuration 34 depicted in FIG. 3, the arterial blood flow enters pipe 36 and continues through and past circulatory pump 38 to enter oxygenator 40. As the blood passes through oxygenator 40, the blood is enriched with oxygen. Oxygenator 40 may also include one or more entrance and exit ports 42a and 42b providing for attachment of a supply line, such as for oxygen or some other material, in any form, gaseous, liquid, or solid. One or more of the ports 42a and 42b may provide an egress useful for a variety of reasons, including for instance, sampling or removing a portion of the blood. Oxygen enriched blood leaves the oxygenator 40 and passes directly into the metabolite transport apparatus or nutritional feeder 44.

As described previously, the nutritional feeder 44 provides nutrition to the blood for use by the fetus or neonate. The nutritional feeder 44 may include one or more intake and/or exit ports 46a, 46b, 46c and 46d which may be used as feed lines and/or exit lines for the nutrients or blood. For instance, nutrients may be fed into each of the nutritional feeder 44 through each of the ports 46a, 46b, 46c and 46d. Alternatively, one or more of the ports, for instance, port 46d, may be used as an egress for the blood. As described with respect to the blood oxygenator 40, the egress may be used for a variety of reasons, including sampling or removing a portion of the blood. Exit 48 provides a simulated arterial return for the blood.

The heater and thermostat, or other temperature regulation apparatus (not shown), may be provided along with or part of the arrangement 34 for the blood oxygenator 40 and nutritional feeder 44. In this way, the temperature of the blood flowing through the arrangement 34 of the blood oxygenator 40 and nutritional feeder 44 may be thermally regulated to simulate the thermal regulation that is inherently provided by maternal blood.

Thus, the blood oxygenation and nutritional system 34 of FIG. 3 may be used in conjunction or as part of a fetus or neonate life support apparatus 2 as provided in FIG. 1, or alternatively the blood oxygenation and nutritional system may be used alone or in combination with various other devices and/or systems.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. An apparatus for supporting a neonate or a fetus, said apparatus comprising:
a fluid tank, said fluid tank being at least partially filled with a simulated amniotic fluid, said simulated amniotic fluid being one that mimics the action of amniotic fluid in a womb;
a dialysis system, wherein said dialysis system is configured to remove waste from said simulated amniotic fluid;
an oxygenator, said oxygenator being configured to oxygenate blood circulating through the neonate or fetus; and
a nutritional feeder, said nutritional feeder being configured to deliver at least one nutrient to said neonate or fetus.

2. The apparatus of claim 1, wherein said oxygenator is further configured to remove waste from said blood.

3. The apparatus of claim 1, further comprising a fluid handling system, wherein said oxygenator and said nutritional feeder are contained within said fluid handling system.

4. The apparatus of claim 1, further comprising a circulatory pump, said circulatory pump being configured to circulate blood through at least one of said oxygenator and said nutritional feeder.

5. The apparatus of claim 4, wherein said circulatory pump is in fluid communication with an umbilical vein and an umbilical artery of said neonate or fetus.

6. The apparatus of claim 1, further comprising a connection apparatus configured to provide a connection with an umbilical vein of said neonate or said fetus and a connection with an umbilical artery of said neonate or fetus.

7. A method of sustaining the life of a neonate or fetus outside of a womb, said method comprising the steps of:
placing the neonate or fetus in a life support system, wherein said life support system includes a tank at least partially filled with a simulated amniotic fluid, and
providing the fetus or neonate with life support sufficient to sustain the life of the neonate or fetus.

8. The method of claim 7, wherein said step of providing the fetus or neonate with life support comprises oxygenating blood circulating through said neonate or fetus using an oxygenator.

9. The method of claim 7, wherein said step of providing the fetus or neonate with life support comprises delivering one or more nutrients to blood circulating through said neonate or fetus using a nutritional feeder.

10. The method of claim 7, wherein said step of providing the fetus or neonate with life support comprises removing waste products from one or both of said simulated amniotic fluid and blood circulating through said neonate or fetus.

11. The method of claim 7, wherein said step of providing the fetus or neonate with life support further comprises the step of pumping blood through one or both of an umbilical artery and an umbilical vein of said fetus or neonate.

12. A connection apparatus for providing a connection to one or both of an umbilical artery and an umbilical vein, said connection apparatus comprising:
an arterial element, said arterial element being configured to be connected to an umbilical artery; and
a venous element, said venous return element being configured to be connected to a venous artery.

13. The connection apparatus of claim 12, wherein said connection apparatus further comprises a circulatory pump configured to cause fluid to flow through one or both of the arterial intake element and the venous return element.

14. A fluid handling system, said fluid handling system comprising:
a nutritional feeder, said nutritional feeder being configured to deliver at least one nutrient to a fluid; and
an oxygenator, said oxygenator being configured to oxygenate said fluid, wherein said fluid handling system is configured for connection to one or both of an umbilical artery and an umbilical vein.

15. The fluid handling system of claim 14, further comprising an apparatus to remove waste from said fluid, wherein said apparatus to remove waste from said fluid is associated with said oxygenator.

16. The fluid handling system of claim 14, further comprising a dialysis system, wherein said dialysis system is configured to remove waste from a second fluid.

17. The fluid handling system of claim 14, further comprising a circulatory pump configured to cause fluid to flow through at least a portion of said fluid handling system.
